(19) United States
(12) Patent Application Publication
(10) Pub. No.: US 2025/0099710 A1
(43) Pub. Date: Mar. 27, 2025

(54) BILATERAL STIMULATION DEVICE

(71) Applicant: Dharma Dr. Technologies, Boise, ID (US)

(72) Inventor: Bradley Mosell, Boise, ID (US)

(21) Appl. No.: 18/473,011

(22) Filed: Sep. 22, 2023

(51) Int. Cl. A61M 21/00; G16H 40/67

(52) U.S. Cl. CPC A61M 21/00; G16H 40/67; A61M 2021/0022; A61M 2021/0027; A61M 2021/0044; A61M 2205/3592

(57) ABSTRACT

Methods, devices, and systems for bilateral stimulation devices are described herein. In some examples, one or more embodiments include a controller comprising a memory, and a processor configured to execute instructions stored in the memory to receive an input to initiate bi-lateral stimulation (BLS) stimuli, generate parameters for the BLS stimuli based on the received input, and cause BLS devices to output the BLS stimuli for a user of the BLS devices according to the generated parameters.

### TECHNICAL FIELD

The present application relates generally to field of bilateral stimulation devices.

### BACKGROUND

EMDR (Eye Movement Desensitization and Reprocessing) is a clinically proven treatment modality for PTSD as well as a wide range of other mental disorders. The treatment consists of an established psychoanalysis protocol alongside administered bilateral stimulation (BLS), which consists of manually stimulating cross hemispheric neural connections through the use of visual, tactile and auditory stimuli. In administering this bilateral stimulation, the clinician often controls the start and stop of the BLS as well as the rate of alternation. BLS is typically administered using a device such as a light bar, tactile stimulators, or headphones. However, in cases where these are unavailable, BLS can be self-administered by having the patient tap along at a cadence directed by the clinician. However, self-administered BLS has been shown to be less effective.

Recently, when overwhelmed by the high demand for mental health services in certain populations, EMDR protocols were developed for group use. EMDR-IGTP (EMDR Integrative Group Treatment Protocol) combines BLS from traditional EMDR with established protocols group therapy protocols. Group therapy has differentiated benefits including greater socialization, reprocessing through empathetic experience, increased communication, teaching individuals how to express their emotions and accept criticism from others. However, due to technological limitations, EMDR-IGTP uses the less effective “self-administered” BLS. Conventional individual use EMDR devices do not allow for the necessary combination of synchronization and differentiation within the group treatment setting. While a clinician can control the start, stop, and rate of alternation, it is critical that the individuals maintain control over the intensity of stimuli and the ability to stop the treatment in order to prevent being overwhelmed, avoid disassociation fear of powerlessness, or perpetuating dependency. New forms of EMDR-IGTP protocols enhanced by improved and extended capabilities enable traditionally successful BLS modalities to be combined with group therapy protocols.

The present disclosure extends the state-of-the-art in EMDR technology using “BLS devices”, a pair of wireless bilateral stimulators that provide both tactile and visual BLS as either an individual set or as part of a larger synchronized network. Individuals may control variables like intensity or joining/exiting the group, while a primary may control individual and group stimulation effects, including by remote control to remote group members, while maintaining synchronization over the speed and intermittency of alternation for all pairs in use by the treatment group. The relationship between synchronization and autonomy and the combination of tactile/visual BLS enables a new form of group EMDR to be administered that is more effective than any previous approaches. Additionally, this control could allow for an EMDR digital therapeutic when the global primary set is controlling alternation speeds along the input of an algorithm or AI control.

### DESCRIPTION OF THE DRAWINGS

FIG. 1A depicts a perspective view of a pair of BLS devices in accordance with one or more embodiments of the present disclosure.

FIG. 1B depicts a bottom view of a BLS device showing charging contacts on the bottom of a BLS device in accordance with one or more embodiments of the present disclosure.

FIG. 2A depicts a storage case for a pair of BLS devices in accordance with one or more embodiments of the present disclosure.

FIG. 2B depicts a front elevation view of the storage case, showing additional case features in accordance with one or more embodiments of the present disclosure.

FIG. 2C depicts a bottom view of the storage case, showing additional case features in accordance with one or more embodiments of the present disclosure.

FIG. 2D depicts a top view of the storage case, showing additional case features in accordance with one or more embodiments of the present disclosure.

FIG. 3 depicts a perspective view of the storage case showing a pair of BLS devices in a charging configuration in accordance with one or more embodiments of the present disclosure.

FIG. 4A depicts a pair of BLS devices utilized by a user in accordance with one or more embodiments of the present disclosure.

FIG. 4B depicts two pairs of BLS devices utilized by different users in accordance with one or more embodiments of the present disclosure.

FIG. 4C depicts a pair of BLS devices utilized by a user, the pair of BLS devices being in communication with a portable control device in accordance with one or more embodiments of the present disclosure.

FIG. 4D depicts two pairs of BLS devices utilized by different users, the pairs of BLS devices being in communication with a portable control device in accordance with one or more embodiments of the present disclosure.

FIG. 4E depicts a pair of BLS devices utilized by a user, the pair of BLS devices being in communication with a portable control device that is in further communication with a remote portable control device in accordance with one or more embodiments of the present disclosure.

FIG. 4F depicts multiple pairs of BLS devices utilized by multiple users in communication with respective portable control devices that are further in communication with a remote portable control device in accordance with one or more embodiments of the present disclosure.

FIG. 5 depicts a user interface of a portable control device during use of BLS devices in accordance with one or more embodiments of the present disclosure.

FIG. 6 depicts a BLS device having a vibration mechanism, an audio output mechanism, a light mechanism, and a controller in accordance with one or more embodiments of the present disclosure.

### DETAILED DESCRIPTION

Embodiments of the present disclosure relate to “BLS devices”, otherwise known in the art as “tappers” and the like. A “BLS device” may refer to a device or a pair of devices for providing BLS. Providing BLS can be utilized during administration of an EMDR treatment for a user, with two “BLS devices” forming a pair of matched BLS devices. Such devices are typically handheld, each capable of administering BLS stimuli by actuating vibrational, audible, and/or visual affects perceptible to the user as described herein.

In general, BLS devices constitute an autonomous pair of devices that may send and receive information between the pair of BLS devices to affect an EMDR treatment. In some embodiments, the “primary” device may control the frequency of alternation and a “secondary” device may control the intensity of vibration. In some implementations, the BLS devices can be turned on for the primary-secondary operation of the BLS devices to function, and either BLS device can be turned off independently to end its operation.

In some embodiments, the functionality of a BLS device pair may allow for the creation of synchronized BLS device groups that can operate locally on an individual set of BLS devices. A secondary BLS device in a group may control the intensity of vibration in each autonomous pair, creating a differentiated experience across at least one dimension of tactile or visual stimulation. A differentiated experience, for example, may accommodate varying levels of individual sensitivity needs or to avoid potential overstimulation of the user of the BLS device pair according to the user's experience.

In some implementations, a “primary” BLS device refers to one BLS device of a pair of BLS devices, or a BLS device of more than one pair of BLS devices that communicates with and controls at least one other BLS device. Additionally, a “global primary” BLS device refers to one BLS device of a pair of BLS devices that communicates with and controls multiple sets of BLS devices. The primary BLS and/or global primary BLS device may control time synchronization, alternation intervals, intensity of the tactile experience, and other functions. As more fully described below, a primary BLS device and/or global primary BLS device may communicate by wired or wireless means, including Bluetooth™, near-field communications (NFC), TCP/IP, IoT, or other uni- or bi-directional communications protocols. The primary BLS device may communicate wirelessly with a secondary BLS device and/or a portable control device. Additionally, the global primary BLS device may communicate wirelessly with multiple sets of BLS devices, and/or a portable control device.

In the disclosure herein, “settings” refer to the parameters of the BLS device, a pair of BLS devices, a primary BLS device, global primary BLS device, multiple sets of BLS devices, or control settings of a portable control device. Settings may be fixed or variable parameters that may control time synchronization, alternation intervals, intensity of the tactile experience, and other functions of a BLS device, BLS device pair, primary or secondary BLS device, or global primary BLS device. Settings may also include user information, identification information, operational information and parameters and the like. Settings may be stored by a single BLS device, a pair of BLS devices, a primary BLS device, global primary BLS device, or by a local or portable control device, which may store user or group settings for one or more BLS devices or tap pairs. A primary BLS device may in turn be controlled by a global primary BLS device from a local or remote location. A primary BLS device may serve as a global primary BLS device by synchronizing with other primary BLS devices. The global primary BLS device may in turn control alternation speed or other settings for all pairs by overriding the primary BLS device of any one or a set of pairs of BLS devices.

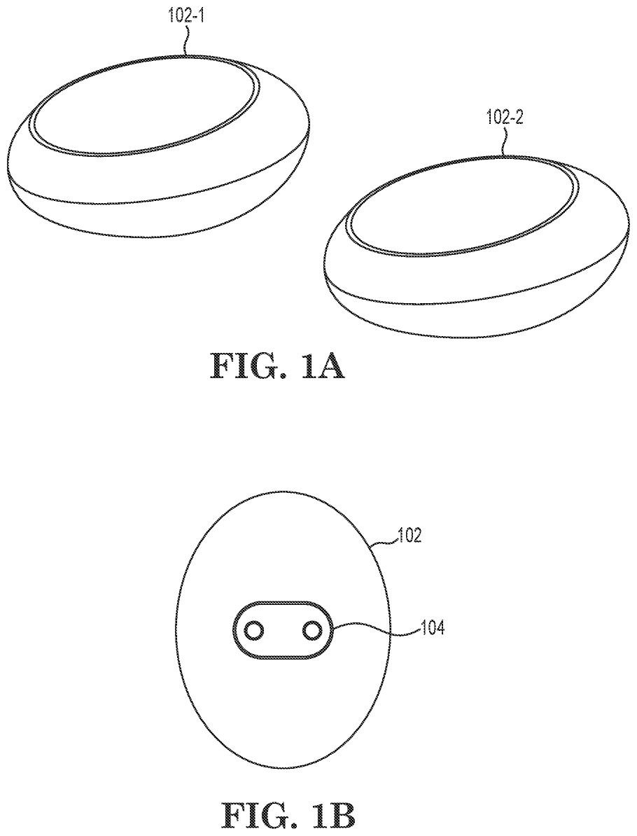

FIG. 1A depicts a perspective view of a pair of BLS devices in accordance with one or more embodiments of the present disclosure. BLS devices 102-1 and 102-2 can function as a “left” BLS device 102-1 and “right” BLS device 102-2 for administering an EMDR treatment. Herein “right” and “left” referring to one BLS device of a BLS device pair are referring to the hand or palm of the user, however, as embodiments described below will demonstrate, the administering of an EMDR treatment may include handheld BLS devices as shown in FIG. 1A or devices attached or held by other means with similar functionality and affect being used to apply EMDR treatment to the user according to the methods herein.

Functionality of each BLS device may include outputting a tactile vibration to the user via vibration mechanism, outputting a visual stimulation via a light mechanism, and/or in some embodiments, outputting an audible stimulation via an audio output mechanism. For example, the vibration mechanism may be a mechanical device that can output oscillations about an equilibrium point, such as a motor having an unbalanced/off-centered weight for providing tactile effects as BLS stimuli. Additionally, the visual stimulation may be provided by one or more light-emitting diodes (LEDs) for providing visual effects as BLS stimuli. Further, the audio output mechanism may be a speaker that can output audio effects as BLS stimuli.

FIG. 1B depicts a bottom view of a BLS device 102 showing charging contacts 104 on the bottom of the BLS device 102 in accordance with one or more embodiments of the present disclosure. As shown in FIG. 1B, in one implementations, each BLS device underside includes contacts 104 for recharging an internal, rechargeable battery while in contact with a corresponding charging device.

In some implementations, the BLS device 102 may house a printed circuit board (PCB). The PCB housed therein may include a processor and a memory for storing data and instructions for performing operations and functionality of the BLS device 102. Instructions performed by the processor may be configured to implement a Boolean logic controller for initialization, charging, and to perform the BLS stimuli and/or EMDR functions as described in detail herein. The PCB may be configured to implement a Boolean logic state machine for control of BLS device functions, such as initialization, charging, standby modes, communications, configuration, and operations in various modes. The PCB may be based, in some embodiments, on the ARDUINO™ hardware and software system, which may be integrated with, or integrate the functions of the BLS device 102. Pressure sensitive actuators may be configured to interact with the PCB to allow user input control of settings of the BLS device 102 and to control operations of the BLS device 102. Operation of the BLS device 102 and BLS device pairs are described in detail below. In some embodiments, each BLS device 102 may include a wireless radio device that can transmit and receive communication signals to perform one or more functions of the BLS device 102, a pair of BLS devices, or a set of BLS device pairs under one or more modes of operation.

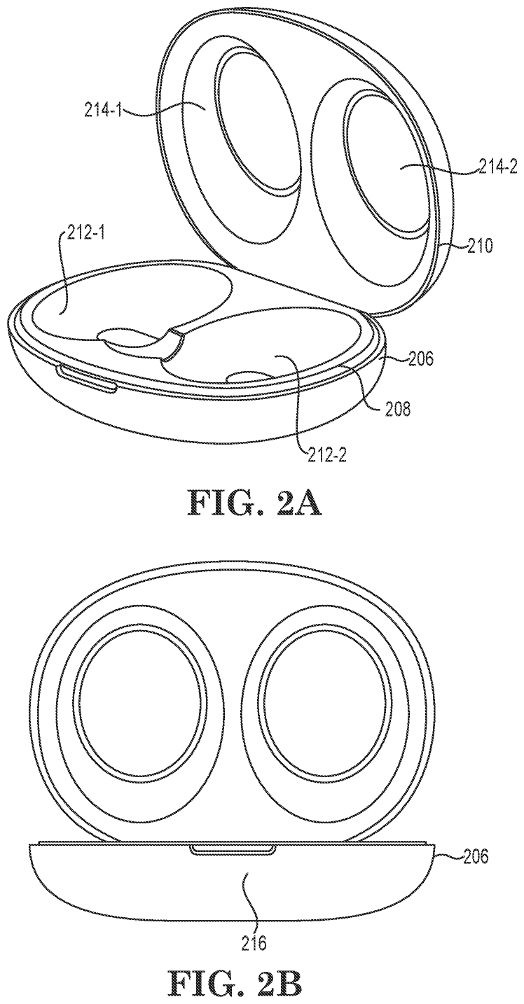

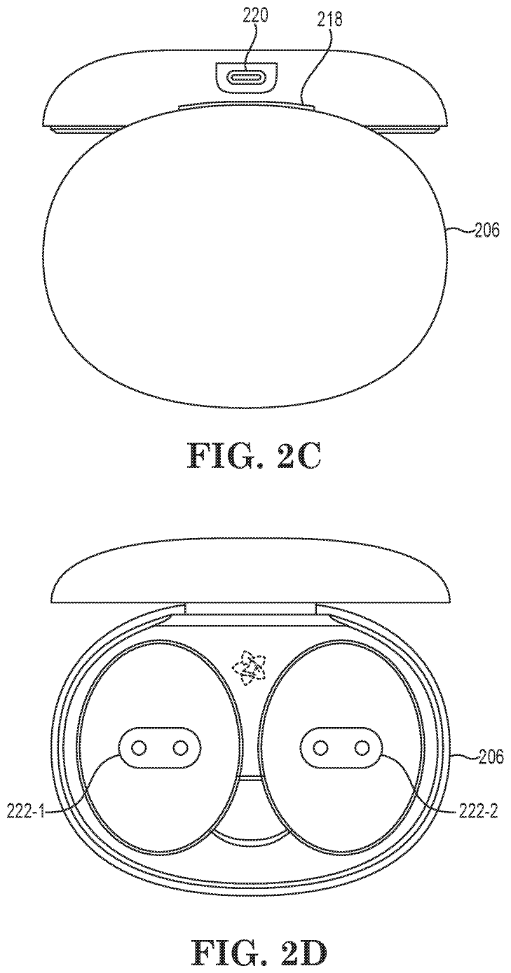

FIG. 2A depicts a storage case 206 for a pair of BLS devices in accordance with one or more embodiments of the present disclosure. The storage case 206, as shown in the open position of FIG. 2A, provides a base 208 and a cover 210 pivotably attached to base 208. Base 208 may include BLS device wells 212-1 and 212-2 that are form-fitted to receive BLS devices (e.g., BLS devices 102-1 and 102-2, respectively) for charging and/or storage.

Cover 210 of storage case 206 provides form-fitted case wells 214-1 and 214-2 aligned with base wells 212-1 and 212-2, respectively, for secure retention of each of a pair BLS devices in wells 212-1 and 212-2 when cover 210 is closed. Base 208 may include a cover retention mechanism and an operational indicator. The operational indicator can be, for example, an LED indicator.

FIG. 2B depicts a front elevation view of the storage case, showing additional case features in accordance with one or more embodiments of the present disclosure. As illustrated in FIG. 2B, the base 208 can include an operational indicator 216. The operational indicator 216 can be an LED that can display different colors based on the operation of the BLS devices. For example, the operational indicator 216 can be green when the BLS devices are fully charged, red when the BLS devices are in operation (e.g., providing BLS stimuli) and not located in the storage case 206, etc.

FIG. 2C depicts a bottom view of the storage case 206, showing additional case features in accordance with one or more embodiments of the present disclosure. For example, the storage case 206 can include a hinge mechanism 218 and a charging port 220. In some implementations, charging port 220 may provide power to the storage case 206 for charging the BLS devices while stored in the BLS device wells.

FIG. 2D depicts a top view of the storage case 206, showing additional case features in accordance with one or more embodiments of the present disclosure. For example, as shown in FIG. 2D, charging contacts 222-1 and 222-2 can receive and align with the charging contacts (e.g., charging contacts 104) of the BLS devices. When such a connection is made, the storage case 206 can provide power to charge the BLS devices.

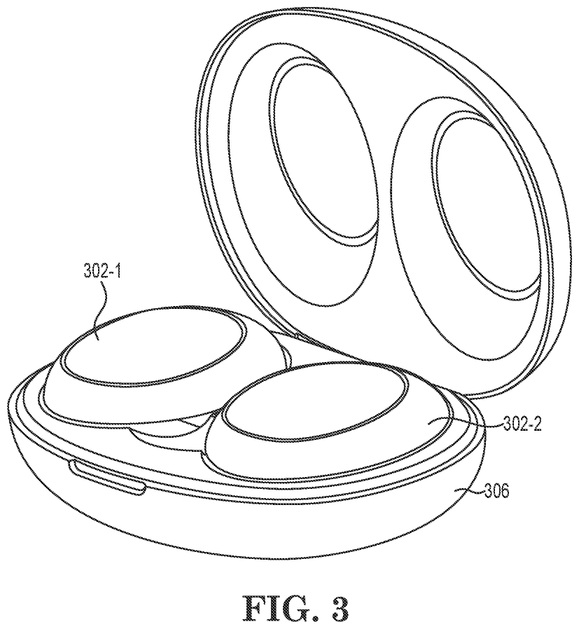

FIG. 3 depicts a perspective view of the storage case 306 showing a pair of BLS devices 302-1 and 302-2 in a charging configuration in accordance with one or more embodiments of the present disclosure. When the BLS devices 302-1 and 302-2 are received into the storage case 306, the charging operation of the BLS devices 302-1 and 302-2 may be initiated upon contact of the charging port of each BLS device 302-1 and 302-2 with the charging contacts in each the wells of the storage case 306.

Additionally, when the BLS devices 302-1 and 302-2 are removed from the storage case 306 for operation, charging operations of the BLS devices 302-1 and 302-2 can cease. Accordingly, a user can then utilize the BLS devices 302-1 and 302-2 for BLS stimuli. Such BLS stimuli may be provided in order to calm the user, utilized as part of a BLS session (e.g., that may be part of an EMDR therapy), etc. Such BLS sessions can be pre-recorded BLS sessions or real-time BLS sessions. The real-time BLS sessions may be single sessions with a therapist, or group sessions with a therapist and other users. Such examples are further described herein.

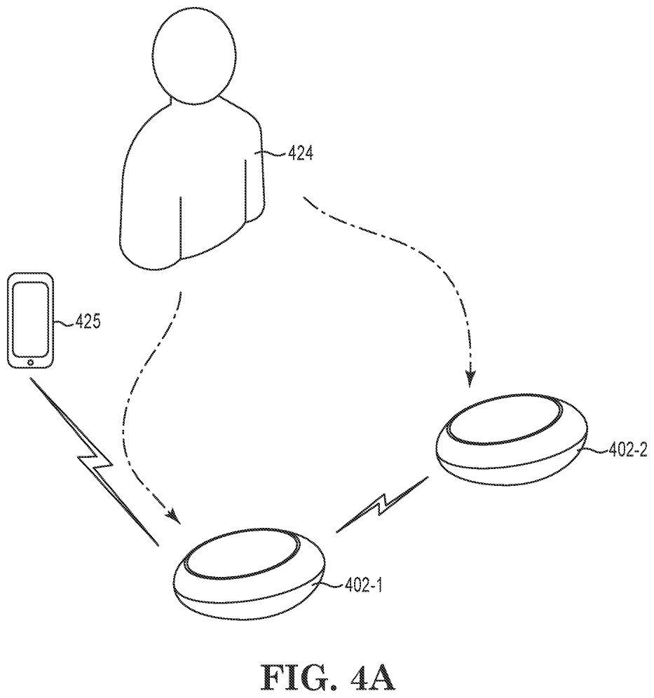

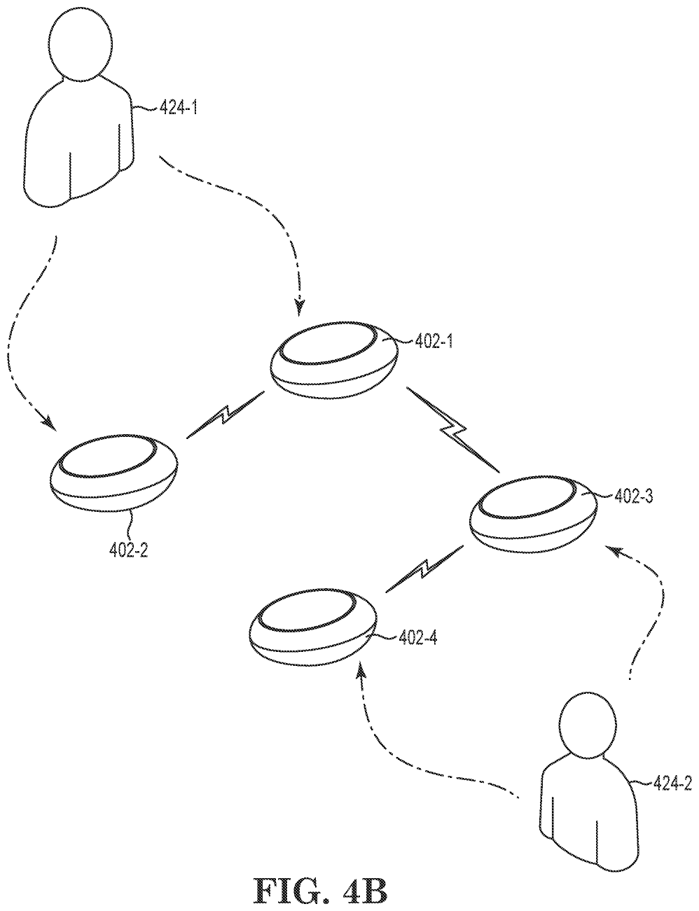

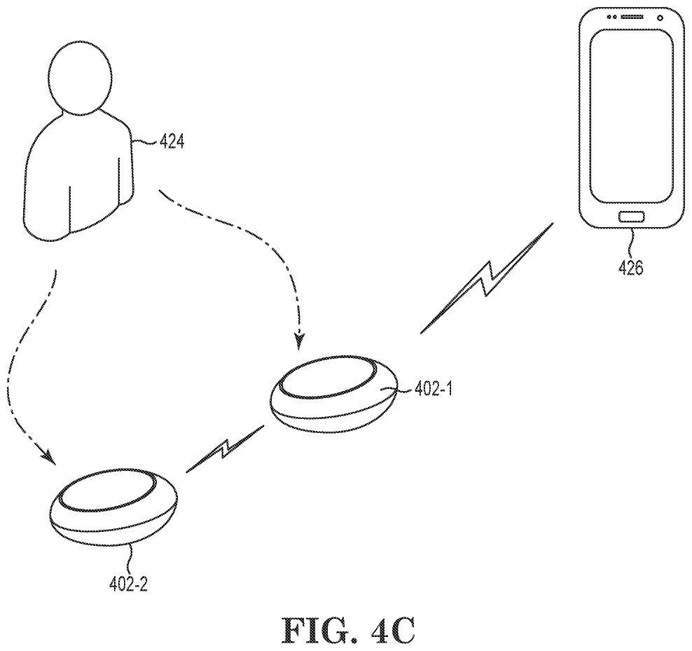

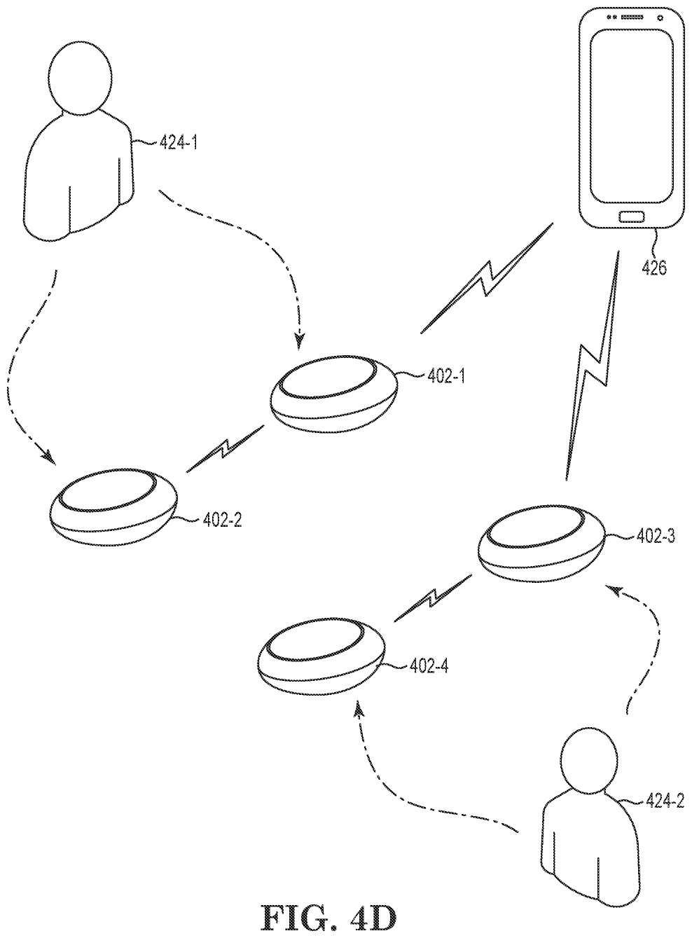

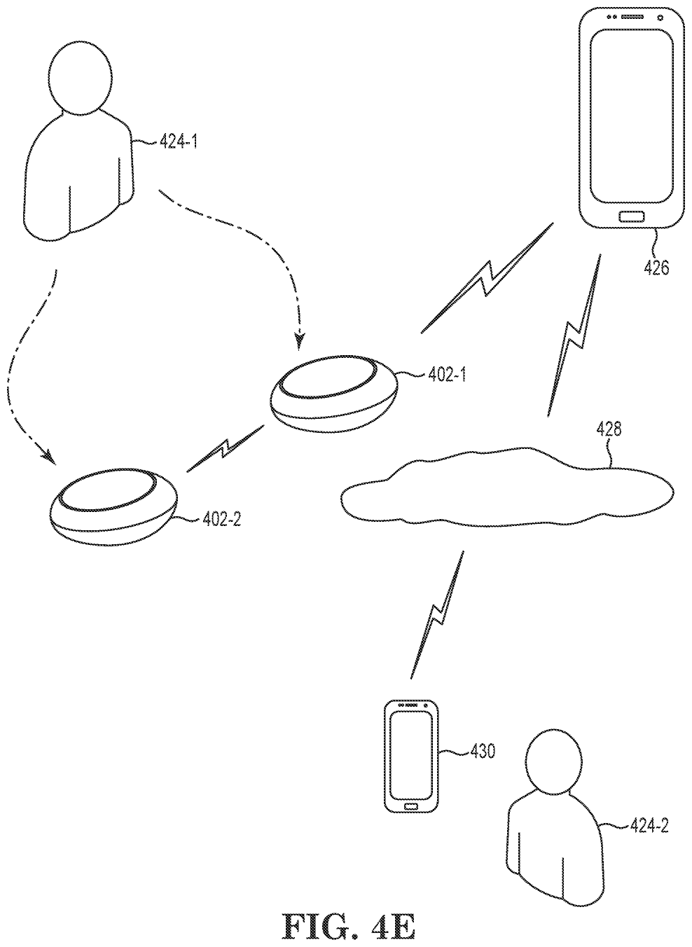

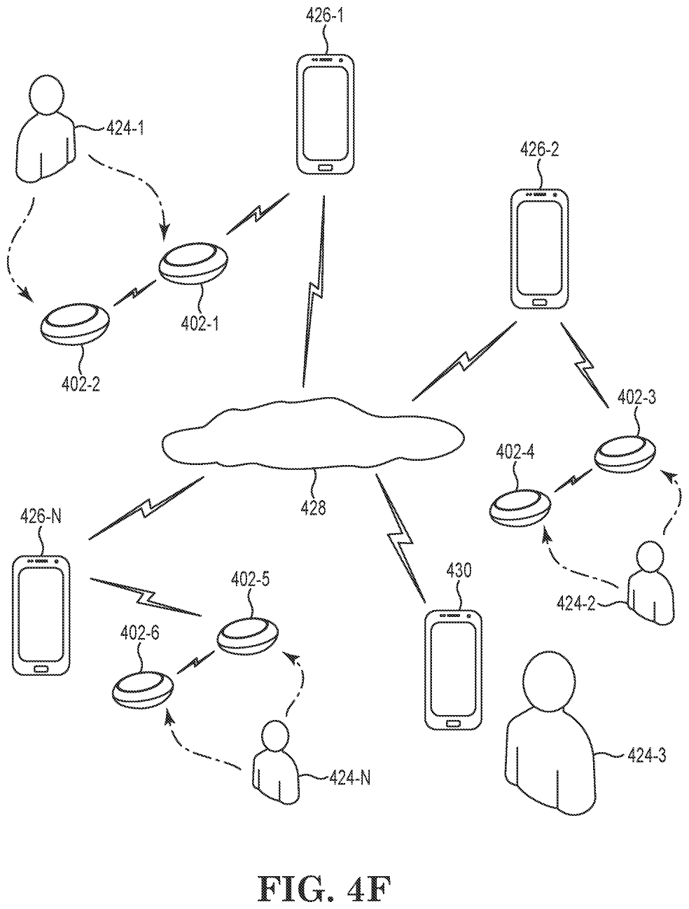

FIG. 4A depicts a pair of BLS devices 402-1 and 402-2 utilized by a user 424 in accordance with one or more embodiments of the present disclosure. The BLS devices 402-1 and 402-2 can be connected via a wireless connection.

In order to administer BLS stimuli, the user 424 can power on the BLS devices 402-1 and 402-2, and the BLS devices 402-1 and 402-2 can connect to each other. Powering on the BLS devices 402-1 and 402-2 may occur automatically as the user 424 removes the BLS devices 402-1 and 402-2 from the storage case, in response to the user 424 pressing a power button (e.g., not illustrated on the BLS devices 402-1 and 402-2), etc.

As illustrated in FIG. 4A, a single user 424 may utilize the BLS devices 402-1 and 402-2. In order to utilize the BLS devices 402-1 and 402-2, the user 424 can provide an input to a controller. The controller can be located in either one of the BLS devices 402-1 and 402-2.

Accordingly, the controller can receive the input to initiate BLS stimuli. The input from the user 424 can cause the controller to generate parameters for the BLS stimuli based on the received input. For example, the user can provide parameters such as a time synchronization between BLS devices 402-1 and 402-2, an alternation interval for the BLS stimuli, and/or an intensity level of the BLS stimuli.

Although the controller is described above as receiving an input from the user 424, embodiments of the present disclosure are not so limited. For example, the controller can receive an input from a portable control device, a remote portable control device, and/or from other BLS devices, as is further described herein.

The user can provide the input to the BLS devices 402-1 and 402-2 via a touch-sensitive (e.g., pressure sensitive) interface. For example, one or both of the BLS devices 402-1 and 402-2 can include a touch-sensitive surface that a user can provide inputs to, and the controller can receive such inputs.

Accordingly, the controller can cause the BLS devices 402-1 and 402-2 to output BLS stimuli for the user 424 of the BLS devices 402-1 and 402-2 according to the generated parameters. Such BLS stimuli can include, for instance, tactile stimulation, visual stimulation, and/or audible stimulation. That is, the controller can cause the BLS devices 402-1 and 402-2 to alternately output tactile stimulation via a vibration mechanism of the BLS devices 402-1 and 402-2 according to the generated parameters, cause the BLS devices 402-1 and 402-2 to alternately output visual stimulation via a light mechanism of the BLS devices 402-1 and 402-2 according to the generated parameters, and/or cause the BLS devices 402-1 and 402-2 to alternately output audible stimulation via an audio output mechanism of the BLS devices 402-1 and 402-2 according to the generated parameters.

For example, the BLS devices 402-1 and 402-2 can operate to affect an EMDR experience on the user 424 under single-user control. In some embodiments, the BLS devices 402-1 and 402-2 alternately apply the BLS for an EMDR therapy to the user 424 through vibrational, visual, and/or audible effects automatically controlled and/or controlled by user interaction with the BLS devices 402-1 and 402-2.

For example, BLS device 402-1 may be the primary BLS device of the BLS devices 402-1 and 402-2, controlling the synchronization, alternation intervals, and/or intensity of the tactile experience by communicating wirelessly with the secondary BLS device 402-2. Additionally, while the BLS device 402-1 is described as being the primary BLS device and BLS device 402-2 as the secondary BLS device, embodiments of the present disclosure are not so limited. For example, the BLS device 402-2 can be the primary BLS device and the BLS device 402-1 can be the secondary BLS device.

As illustrated in FIG. 4A, the user 424 may further have a mobile device 425. The mobile device 425 can be, for example, a device that can be carried and/or worn by a user. For example, the mobile device 425 can be a phone (e.g., a smart phone), smart glasses, a wrist-worn device (e.g., a smart watch), and/or any other device that can be carried and/or worn by a user.

The mobile device 425 can be a device that can measure biometric information about the user 424. Biometric information can be information about an anatomical and/or physiological characteristic of a user. For example, the mobile device 425 can measure biometric information about the user 424, including retinal information, facial expressions, heart rate, heart rate variability, respiratory information, blood pressure information, voice information, etc.

For example, prior to beginning a BLS session, the mobile device 425 may gather certain biometric information, such as heart rate, of the user 424 and transmit the biometric information to the controller. The controller can receive the biometric information about the user 424 from the mobile device 425, and generate the parameters for the BLS stimuli based on the biometric information.

In some examples, the mobile device 425 may gather biometric information during a BLS session. For example, the mobile device 425 may determine the initial heart rate of the user 424 prior to beginning the BLS session, and then determine an updated heart rate while the BLS session is ongoing. The controller can compare the updated heart rate to the initial heart rate and determine, based on the comparison, that the change in heart rate has increased beyond a threshold heart rate level. Accordingly, the controller can generate updated parameters based on the increase in heart rate level, and cause the BLS devices 402-1, 402-2 to output updated BLS stimuli according to the updated parameters.

While the controller is described above as generating updated parameters based on an increase in heart rate level exceeding a threshold heart rate level amount, embodiments of the present disclosure are not so limited. For example, the controller can generate updated parameters based on a decrease in heart rate level exceeding a threshold heart rate level amount. Such thresholds can associated with the same change amount in heart rate level, or can be associated with different amounts of change in heart rate level.

Additionally, while the controller is described above as generating updated parameters based on heart rate level, embodiments of the present disclosure are not so limited. For example, the controller can generate updated parameters based on any other type of biometric information about the user 424.

Although not illustrated in FIG. 1, the BLS devices 402-1, 402-2 can be connected (e.g., wirelessly) to a remote computing device, such as a cloud server (e.g., either directly, or through a portable control device). The remote computing device can store data from other BLS devices. For example, the remote computing device can receive data, including data about users of the other BLS devices (e.g., age, sex, condition, biometric data, etc.). The remote computing device can utilize artificial intelligence (AI) to determine patterns in the stored data from other BLS devices to provide parameters for BLS stimuli that may be useful for the specific condition of the user 424. For example, the BLS device 402-1 may transmit biometric information to the remote computing device, and the remote computing device can transmit parameters generated by the remote computing device for BLS stimuli based on a comparison of the user 424's biometric information with previously stored information in the remote computing device for use by the user 424.

In some examples, the mobile device 425 can query the user 424 for various questions about the user's mental state. The user 424 may provide answers to the questions by providing inputs to the mobile device 425, while also simultaneously transmitting biometric information. The queries can be modeled after current and/or proposed EMDR protocols, and can be generated by AI based on the users biometric information, past answers from other BLS device users, etc. Such answers, along with the biometric information, can be transmitted to the remote computing device, where the remote computing device can perform analysis on the answers and biometric information with various AI models to generate parameters for the BLS stimuli. This process may be repeated to update parameters for the BLS stimuli for the user 424. For example, as the user 424 experiences the BLS stimuli, additional queries may be presented to the user 424, again via the mobile device 425. As answers to the additional queries may change, the remote computing device can generate different/updated BLS stimuli that can be transmitted to the BLS devices 402-1, 402-2. Accordingly, under such an approach, an AI assisted protocol for queries/questionnaires can be developed based on user answers for a large population of BLS device users, which can allow for the modification of EMDR protocols and specific targeting of particular BLS stimuli based on individual users.

FIG. 4B depicts two pairs of BLS devices utilized by different users in accordance with one or more embodiments of the present disclosure. For example, user 424-1 can be utilizing BLS devices 402-1 and 402-2, while user 424-2 can be utilizing BLS devices 402-3 and 402-4.

In some examples, user 424-1 can control the administration of the BLS stimuli for the BLS devices 402-1 and 402-2, as well as the BLS devices 402-3 and 402-4. For example, the BLS device 402-1 can be a primary BLS device that controls the synchronization, alternation intervals, and/or intensity of the tactile experience by communicating wirelessly with the secondary BLS device 402-2, as well as a global primary BLS device that controls the synchronization, alternation intervals, and/or intensity of the tactile experience by communicating wirelessly with the primary BLS device 402-3. The primary BLS device 402-3 can then control the synchronization, alternation intervals, and/or intensity of the tactile experience by communicating wirelessly with the secondary BLS device 402-4.

Therefore, each BLS device pair applies the BLS effect to its respective user through vibrational, visual, and/or audible effects controlled by user interaction with the BLS devices 402-1, 402-2, 402-3, 402-4. For example, BLS devices 402-1 and 402-3 may be the primary BLS devices for each set of BLS devices. Additionally, BLS device 402-1 of user 424-1 may be the global primary BLS device for both pairs of BLS devices, controlling the synchronization, alternation intervals, intensity of the tactile experience for both users 424-1 and 424-2 by communicating wirelessly with the primary BLS device 402-3 of user 424-2, thereby causing both users to share the same BLS and/or EMDR experience.

Alternatively, or in addition to the global primary BLS device 402-1 controlling all settings of both pairs of BLS devices, in some implementations, the secondary BLS devices 402-2, 402-4 may separately controlling the synchronization, alternation intervals, intensity of the tactile experience for each user. It should be understood that while FIG. 4B depicts a two-user multi-user mode, the operation of the multiple BLS device pairs may extend to any number of BLS device pairs in use and controlled by the global primary BLS device for multiple users.

FIG. 4C depicts a pair of BLS devices 402-1, 402-2 utilized by a user 424, the pair of BLS devices 402-1, 402-2 being in communication with a portable control device 426 in accordance with one or more embodiments of the present disclosure. The BLS devices 402-1 and 402-2 can be connected via a wireless connection, and the BLS device 402-1 can be connected to the portable control device 426 via a wireless connection.

Similar to FIG. 4A, a single user 424 may utilize the BLS devices 402-1 and 402-2. In order to utilize the BLS devices 402-1 and 402-2, the user 424 can provide an input to a controller. The controller can be located in the portable control device 426. That is, the user 424 can provide an input to the portable control device 426 (e.g., via a user interface of the portable control device 426).

Accordingly, the controller can receive the input to initiate BLS stimuli. The input from the user 424 can cause the controller to generate parameters for the BLS stimuli based on the received input. For example, the user can provide parameters such as a time synchronization between BLS devices 402-1 and 402-2, an alternation interval for the BLS stimuli, and/or an intensity level of the BLS stimuli.

Accordingly, the controller can cause the BLS devices 402-1 and 402-2 to output BLS stimuli for the user 424 of the BLS devices 402-1 and 402-2 according to the generated parameters. Such BLS stimuli can include, for instance, tactile stimulation, visual stimulation, and/or audible stimulation. That is, the controller can cause the BLS devices 402-1 and 402-2 to alternately output tactile stimulation via a vibration mechanism of the BLS devices 402-1 and 402-2 according to the generated parameters, cause the BLS devices 402-1 and 402-2 to alternately output visual stimulation via a light mechanism of the BLS devices 402-1 and 402-2 according to the generated parameters, and/or cause the BLS devices 402-1 and 402-2 to alternately output audible stimulation via an audio output mechanism of the BLS devices 402-1 and 402-2 according to the generated parameters.

In some examples, the parameters can be generated utilizing information included in the input. For example, the user 424 may input to the portable control device 426 information in order to access a pre-recorded BLs session. Such a pre-recorded BLS session may be recorded by a therapist for an EMDR session, and saved in a remote computing device, such as a server (e.g., a cloud server). The portable control device 426 may retrieve, from the remote computing device, predetermined session data for the pre-recorded BLS session. Such predetermined session data can include information to allow the controller to generate parameters for the BLS stimuli utilizing the predetermined session data.

Accordingly, the controller can cause the BLS devices 402-1, 402-2 to output BLS stimuli for the user 424 according to the generated parameters for the pre-recorded BLS session. That is, the controller can cause the BLS devices 402-1, 402-2 to alternately output tactile stimulation via a vibration mechanism of the BLS devices 402-1 and 402-2 according to the generated parameters for the pre-recorded BLS session, cause the BLS devices 402-1 and 402-2 to alternately output visual stimulation via a light mechanism of the BLS devices 402-1 and 402-2 according to the generated parameters for the pre-recorded BLS session, and/or cause the BLS devices 402-1 and 402-2 to alternately output audible stimulation via an audio output mechanism of the BLS devices 402-1 and 402-2 according to the generated parameters for the pre-recorded BLS session.

The portable control device 426 can be, for example, a smartphone device, a tablet computing device, a laptop computer and the like, according to one embodiment of the invention. In such an operation, the BLS devices 402-1 and 402-2 operate to affect an EMDR experience on the user under control of the portable control device 426. As above, BLS devices 402-1 and 402-2 alternately apply the EMDR effect to the user through vibrational, visual, and/or audible actuations of the BLS devices 402-1 and 402-2. BLS device 402-1 may function as the primary BLS device controlling the synchronization, alternation intervals, intensity of the tactile experience, by communicating wirelessly with the secondary BLS device 402-2. The primary BLS device 402-1 may in turn communicate wirelessly with the portable control device 426 to receive settings for the operation of the BLS devices 402-1 and 402-2 providing the user EMDR experience.

FIG. 4D depicts two pairs of BLS devices utilized by different users 424-1, 424-2, the pairs of BLS devices being in communication with a portable control device 426 in accordance with one or more embodiments of the present disclosure. For example, user 424-1 can be utilizing BLS devices 402-1 and 402-2, while user 424-2 can be utilizing BLS devices 402-3 and 402-4.

In some examples, user 424-1 can control the administration of the BLS stimuli for the BLS devices 402-1 and 402-2, as well as the BLS devices 402-3 and 402-4, via the portable control device 426. For example, the BLS device 402-1 can be a primary BLS device that controls the synchronization, alternation intervals, and/or intensity of the tactile experience by communicating wirelessly with the secondary BLS device 402-2, and the BLS device 402-3 can be a primary BLS device that controls the synchronization, alternation intervals, and/or intensity of the tactile experience by communicating wirelessly with the secondary BLS device 402-4. The primary BLS devices 402-1 and 402-3 can receive inputs from the portable control device 426.

Therefore, each BLS device pair applies the BLS effect to its respective user through vibrational, visual, and/or audible effects controlled by user interaction with the BLS devices 402-1, 402-2, 402-3, 402-4. For example, BLS device 402-1 may be the primary BLS device and BLS device 402-2 may be the secondary BLS device for the device pair for user 424-1, and BLS device 402-3 may be the primary BLS device and BLS device 402-4 may be the secondary BLS device for the device pair for user 424-2. The primary BLS devices 402-1 and 402-3 can control the synchronization, alternation intervals, intensity of the tactile experience for both users 424-1 and 424-2 by communicating wirelessly with the respective secondary devices 402-2 and 402-4, thereby causing both users 424-1 and 424-2 to share the same BLS and/or EMDR experience.

Accordingly, multiple pairs of BLS devices operate to affect an EMDR experience on each user 424-1, 424-2 under control of the portable control device 426. BLS devices 402-1 and 402-2 apply BLS stimuli to user 424-1 and BLS devices 402-3 and 402-4 apply BLS stimuli to user 424-2. The primary BLS devices 402-1 and 402-3 may in turn communicate wirelessly with the portable control device 426 to receive settings for the operation of a pair of BLS devices for providing each user EMDR experience.

Alternatively, or in addition to the portable control device 426 controlling all settings of all pairs of BLS devices, in some implementations, the secondary BLS devices 402-2 and 402-4 may separately control the intensity of the tactile experience for each set of BLS devices held by the respective users 424-1, 424-2. It should be understood that while FIG. 4D depicts a two-user multi-user app mode, the operation of the multiple BLS device pairs may extend to any number of BLS device pairs in use and controlled by the portable control device.

In some examples, the portable control device 426 can transmit inputs to a first controller (e.g., located in BLS device 402-1) and a second controller (e.g., located in BLS device 402-2). The first controller can receive the input to initiate BLS stimuli, and the second controller can receive the input to initiate BLS stimuli. The input can include session data for a BLS session. The first controller can generate parameters for the BLS stimuli using the session data, and the second controller can also generate parameters for the BLS stimuli using the session data. The session data can be for a real-time BLS session. For example, the users 424-1 and 424-2 can intend to join a real-time BLS session. The therapist may utilize the portable control device 426 to transmit the inputs to the respective controllers for BLS devices 402-1 and 402-3.

The respective controllers can, accordingly, cause the BLS devices 402-1 and 402-2 to output BLS stimuli for the user 424-1 according to the generated parameters for the real-time BLS session, and cause the BLS devices 402-3 and 402-4 to output BLS stimuli for the user 424-2 according to the generated parameters for the real-time BLS session. Such an approach may be utilized by, for example, a therapist for in-person group EMDR treatments utilizing BLS stimuli.

FIG. 4E depicts a pair of BLS devices 402-1, 402-2 utilized by a user 424-1, the pair of BLS devices being in communication with a portable control device 426 that is in further communication with a remote portable control device 430 in accordance with one or more embodiments of the present disclosure. The BLS devices 402-1 and 402-2 can be connected via a wireless connection, and the BLS device 402-1 can be connected to the portable control device 426 via a wireless connection.

Similar to FIG. 4C, a single user 424 may utilize the BLS devices 402-1 and 402-2. In order to utilize the BLS devices 402-1 and 402-2, the user 424-2 can provide an input to a controller. For example, the user 424-2 may be a therapist that is remotely controlling administration of a BLS session (e.g., for an EMDR therapy) using BLS devices 402-1 and 402-2. The user 424-2 may provide an input to the remote portable control device 430. The portable control device 426 may be a mobile device utilized by the user 424-1 that is wirelessly connected to the primary BLS device 402-1, and the primary BLS device 402-1 can be wirelessly connected to the secondary BLS device 402-2. The input can be transmitted from the remote portable control device 430 to a remote computing device 428 (e.g., a cloud server, etc.), where the portable control device 426 can retrieve the input and transmit to a controller of the primary BLS device 402-1.

Accordingly, the pair of BLS devices 402-1 and 402-2 can be in communication with the portable control device 426 under the control of the remote portable control device 430. The pair of BLS devices 402-1 and 402-2 operate to affect BLS stimuli (e.g., via an EMDR experience) on the user 424-1 under control of the portable control device 426. BLS devices 402-1 and 402-2 alternately apply the BLS stimuli (e.g., for an EMDR effect) to the user 424-1 through vibrational, audible, and/or visual actuations of the BLS devices 402-1 and 402-2. BLS device 402-1 may function as the primary BLS device controlling the synchronization, alternation intervals, intensity of the tactile experience, by communicating wirelessly with the secondary BLS device 402-2. The primary BLS device 402-1 may in turn communicate wirelessly with the portable control device 426 to receive settings for the operation of the pair of BLS devices 402-1 and 402-2 providing the user EMDR experience. The portable control device 426 may, in turn, be controlled remotely by the remote portable control device 430, which, in some embodiments, provides for a remote user to control the EMDR experience for the user.

FIG. 4F depicts multiple pairs of BLS devices 402 utilized by multiple users 424 in communication with respective portable control devices 426 that are further in communication with a remote portable control device 430 in accordance with one or more embodiments of the present disclosure. In such an approach, a user 424-3 (e.g., a therapist) may control a group BLS session for group EMDR therapy. The user 424-3 may be remotely located from the users 424-1, 424-2, 424-N.

Similar to FIG. 4D, multiple users 424 may utilize BLS devices. For example, user 424-1 may utilize BLS devices 402-1, 402-2, user 424-2 may utilize BLS devices 402-3, 402-4, and user 424-N may utilize BLS devices 402-5, 402-6. In order to utilize the BLS devices in such a group therapy session, the individual users 424-1, 424-2, 424-N may utilize portable control devices 426-1, 426-2, 426-N, respectively. The user 424-3 may be a therapist that is remotely controlling administration of a BLS session (e.g., for an EMDR therapy) using BLS devices 402-1, 402-2, 402-3, 402-4, 402-5, and 402-6. The user 424-3 may provide an input to the remote portable control device 430. The portable control device 426-1 may be a mobile device utilized by the user 424-1 that is wirelessly connected to the primary BLS device 402-1, and the primary BLS device 402-1 can be wirelessly connected to the secondary BLS device 402-2. The input can be transmitted from the remote portable control device 430 to a remote computing device 428 (e.g., a cloud server, etc.), where the portable control device 426-1 can retrieve the input and transmit to a controller of the primary BLS device 402-1.

Accordingly, the pair of BLS devices 402-1 and 402-2 can be in communication with the portable control device 426-1 under the control of the remote portable control device 430. The pair of BLS devices 402-1 and 402-2 operate to affect BLS stimuli (e.g., via an EMDR experience) on the user 424-1 under control of the portable control device 426-1. BLS devices 402-1 and 402-2 alternately apply the BLS stimuli (e.g., for an EMDR effect) to the user 424-1 through vibrational, audible, and/or visual actuations of the BLS devices 402-1 and 402-2. BLS device 402-1 may function as the primary BLS device controlling the synchronization, alternation intervals, intensity of the tactile experience, by communicating wirelessly with the secondary BLS device 402-2. The primary BLS device 402-1 may in turn communicate wirelessly with the portable control device 426-1 to receive settings for the operation of the pair of BLS devices 402-1 and 402-2 providing the user EMDR experience. The portable control device 426-1 may, in turn, be controlled remotely by the remote portable control device 430, which, in some embodiments, provides for a remote user to control the EMDR experience for the user 424-1.

Additionally, the portable control device 426-2 may be a mobile device utilized by the user 424-2 that is wirelessly connected to the primary BLS device 402-3, and the primary BLS device 402-3 can be wirelessly connected to the secondary BLS device 402-4. The input can be transmitted from the remote portable control device 430 to a remote computing device 428 (e.g., a cloud server, etc.), where the portable control device 426-2 can retrieve the input and transmit to a controller of the primary BLS device 402-3.

Therefore, the pair of BLS devices 402-3 and 402-4 can be in communication with the portable control device 426-2 under the control of the remote portable control device 430. The pair of BLS devices 402-3 and 402-4 operate to affect BLS stimuli (e.g., via an EMDR experience) on the user 424-2 under control of the portable control device 426-2. BLS devices 402-3 and 402-4 alternately apply the BLS stimuli (e.g., for an EMDR effect) to the user 424-2 through vibrational, audible, and/or visual actuations of the BLS devices 402-3 and 402-4. BLS device 402-3 may function as the primary BLS device controlling the synchronization, alternation intervals, intensity of the tactile experience, by communicating wirelessly with the secondary BLS device 402-4. The primary BLS device 402-3 may in turn communicate wirelessly with the portable control device 426-2 to receive settings for the operation of the pair of BLS devices 402-3 and 402-4 providing the user EMDR experience. The portable control device 426-2 may, in turn, be controlled remotely by the remote portable control device 430, which, in some embodiments, provides for a remote user to control the EMDR experience for the user 424-2.

Further, the portable control device 426-N may be a mobile device utilized by the user 424-N that is wirelessly connected to the primary BLS device 402-5, and the primary BLS device 402-5 can be wirelessly connected to the secondary BLS device 402-6. The input can be transmitted from the remote portable control device 430 to a remote computing device 428 (e.g., a cloud server, etc.), where the portable control device 426-N can retrieve the input and transmit to a controller of the primary BLS device 402-5.

Lastly, the pair of BLS devices 402-5 and 402-6 can be in communication with the portable control device 426-N under the control of the remote portable control device 430. The pair of BLS devices 402-5 and 402-6 operate to affect BLS stimuli (e.g., via an EMDR experience) on the user 424-N under control of the portable control device 426-N. BLS devices 402-5 and 402-6 alternately apply the BLS stimuli (e.g., for an EMDR effect) to the user 424-N through vibrational, audible, and/or visual actuations of the BLS devices 402-5 and 402-6. BLS device 402-5 may function as the primary BLS device controlling the synchronization, alternation intervals, intensity of the tactile experience, by communicating wirelessly with the secondary BLS device 402-6. The primary BLS device 402-5 may in turn communicate wirelessly with the portable control device 426-N to receive settings for the operation of the pair of BLS devices 402-5 and 402-6 providing the user EMDR experience. The portable control device 426-N may, in turn, be controlled remotely by the remote portable control device 430, which, in some embodiments, provides for a remote user to control the EMDR experience for the user 424-N.

Accordingly, the portable control devices 426-1, 426-2, 426-N can transmit inputs to a first controller (e.g., located in BLS device 402-1), a second controller (e.g., located in BLS device 402-3), and an N'th controller (e.g., located in BLS device 402-N). The first controller, the second controller, and the third controller can all receive the input to initiate BLS stimuli. The input can include session data for a BLS session. The first controller, second controller, and the third controller can all generate parameters for the BLS stimuli using the session data. The session data can be for a real-time BLS session. For example, the users 424-1, 424-2, and 424-N can intend to join a real-time BLS session. The therapist may utilize the portable control devices 426-1, 426-2, 426-N to transmit the inputs to the respective controllers for BLS devices 402-1, 402-3, and 402-5.

The respective controllers can, accordingly, cause the BLS devices 402-1 and 402-2 to output BLS stimuli for the user 424-1 according to the generated parameters for the real-time BLS session, cause the BLS devices 402-3 and 402-4 to output BLS stimuli for the user 424-2 according to the generated parameters for the real-time BLS session, and cause the BLS devices 402-5 and 402-6 to output BLS stimuli for the user 424-N according to the generated parameters for the real-time BLS session. Such an approach may be utilized by, for example, a remotely located therapist for group EMDR treatments utilizing BLS stimuli.

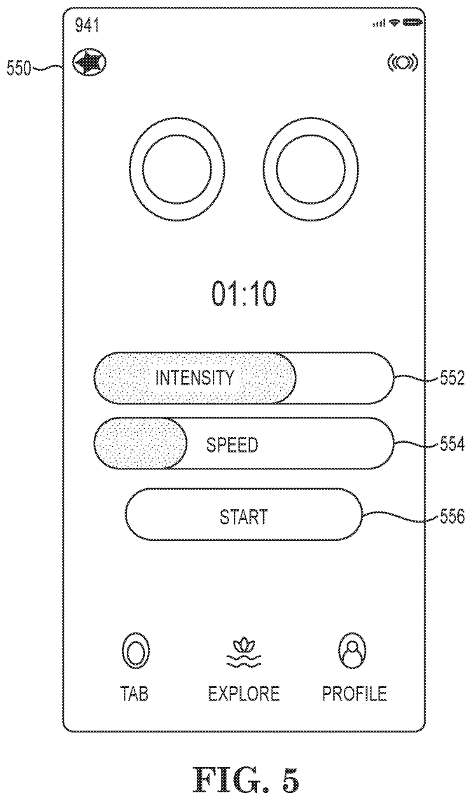

FIG. 5 depicts a user interface 550 of a portable control device during use of BLS devices in accordance with one or more embodiments of the present disclosure. A user of the portable control device can cause the BLS devices to output BLS stimuli utilizing the user interface 550, as is further described herein.

A user utilizing the portable control device having the user interface 550 can provide an input to the user interface 550. Such an input can include session data for a BLS session and can be transmitted to a controller of the BLS devices to cause the controller of the BLS devices to generate parameters for BLS stimuli based on the session data. The input can then cause the BLS devices to output BLS stimuli for a user of the BLS devices, as described above.

For example, a user can provide an input to the user interface 550 to control an intensity of the BLS stimuli via the slider 552. The user can increase the intensity by sliding the slider 552 to the right (e.g., as oriented in FIG. 5), and decrease the intensity by sliding the slider 552 to the left (e.g., as oriented in FIG. 5). Accordingly, the intensity slider 552 can set the intensity of the BLS stimuli of the BLS devices, can modify the intensity of the BLS stimuli of the BLS devices if the output of the BLS stimuli is currently underway, etc.

Additionally, a user can provide an input to the user interface 550 to control an alternation interval speed of the BLS stimuli via the slider 554. The user can increase the alternation interval by sliding the slider 554 to the right (e.g., as oriented in FIG. 5), and decrease the alternation interval by sliding the slider 554 to the left (e.g., as oriented in FIG. 5). Accordingly, the alternation interval speed slider 554 can set the alternation interval of the BLS stimuli of the BLS devices, can modify the alternation interval of the BLS stimuli of the BLS devices if the output of the BLS stimuli is currently underway, etc.

When such session data is set, the input can be transmitted to the controller of the BLS devices in response to the user interface 550 receiving an input at the start button 556 (e.g., being pressed). For example, the user of the portable control device can provide an input by pressing the start button 556 on the user interface 550, which can transmit the input to the controller. Further, in some examples, although not illustrated in FIG. 5 for clarity and so as not to obscure embodiments of the present disclosure, while a BLS session is underway, the user interface 550 may illustrate an “update” button, which can transmit updated session data (e.g., updated alternation intervals, updated intensity levels, etc.) to the controller when such an update button is pressed (e.g., a user presses the update button displayed on the user interface 550).

Although not illustrated in FIG. 5 for clarity and so as not to obscure embodiments of the present disclosure, the user interface 550 can display options for selecting and/or modifying the particular BLS stimuli to be output. For example, the user interface 550 can display options for outputting tactile stimulation, visual stimulation, and/or audible stimulation. A user may select, via the user interface 550, tactile and visual stimulation, and upon transmission of the session data to the controller, cause the BLS devices to output tactile and visual stimulation for a user of the BLS devices. Additionally, a user may further select audible stimulation during the BLS session, and as such, cause transmission of updated session data to the controller to cause the BLS devices to further output audible stimulation in addition to the tactile and visual stimulation.

Additionally, although not illustrated in FIG. 5 for clarity and so as not to obscure embodiments of the present disclosure, the user interface 550 can display a unique identifier of the BLS device. For example, the BLS devices currently having session data input for can transmit a unique identifier to the portable control device (e.g., or a remote portable control device), and a user of the portable control device can determine which BLS devices they are setting session data for. Additionally, the user interface 550 can display a list of unique identifiers corresponding to the BLS devices which are in communication with the portable control device. Accordingly, in a setting in which a therapist may be performing a group EMDR therapy, the therapist utilizing the portable control device can utilize the user interface 550 to set session data for the BLS devices in communication with the portable control device. This can allow the therapist to setup BLS devices differently based on requirements of each patient (e.g., user) using the BLS devices. That is, the therapist may select different BLS stimuli, and/or different session data for different parameters for the different BLS devices, allowing the therapist flexibility on how intense each patient may desire the BLS stimuli.

A substantial benefit of the foregoing functionality is the administration of EMDR treatment in a group setting. EMDR treatment in group settings may be advantageously controlled by an individual of the group, for example, a therapist.

For example, a therapist may seek to administer BLS in a group setting. This group of people may or may not have experienced a similar traumatic incident or are at a similar stage in their therapeutic treatment. Each patient is instructed to take out their BLS devices. The therapist can open their portable control device (or remote portable control device) and select all of the BLS device pairs to be controlled. This pairing can create a mesh that synchronizes global control of all the BLS device pairs. The therapist can then select an intensity and speed setting and begin treatment. Patients are instructed to use the bilateral stimulation until they experience a change in their physiology. While treatment is administered, several patients may stop their BLS devices because they had a sudden shift while others continue until the therapist pauses the treatment. This process can be repeated until the end of the session and the mesh is dissolved.

As a further example, a remote instructor may seek to administer bilateral stimulation to a decentralized group setting. This group of people may be local to each other, remote from each other, or some combination thereof. This group of people may or may not have experienced a similar traumatic incident or are at a similar stage in their therapeutic treatment. Each patient can have one or multiple pairs (micro group) of BLS devices paired to a portable control device (or remote portable control device). Using the user interface on this portable control device, the patients can select or enroll into a session that will be controlled by a therapist (locally or remotely located). This therapist can administer the class/session to all of the individuals or groups that have enrolled locally and/or remotely.

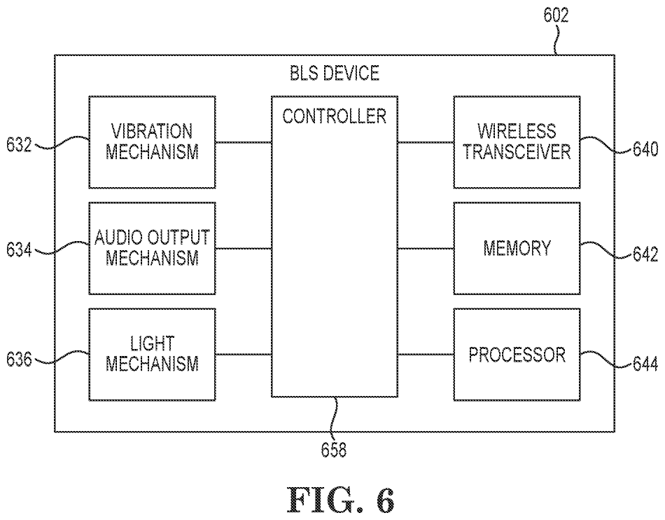

FIG. 6 depicts a BLS device 602 having a vibration mechanism 632, an audio output mechanism 634, a light mechanism 636, and a controller 638 in accordance with one or more embodiments of the present disclosure. The BLS device 602 can further include a wireless transceiver 640, a memory 642, and a processor 644.

In some implementations, controller 638 may comprise a Fanstel BM840 BLE module paired with a TC2050-IDC XLINX-type cable connector for programming and debugging of the controller 638. Digital inputs to the controller 638 of FIG. 6 may include a button switch input for pressure actuation by the user of the BLS device 602, a case charging indicator and a case connected indicator for operation with the BLS device case as previously described. Outputs of the controller 638 may include control of a vibration mechanism 632, such as a motor controlled by pulse width modulation (PWM) for vibratory control of the BLS device 602 effects, an audio output mechanism 634, such as one or more speakers for audible outputs of BLS device operation and for the control of audible effects, and a light mechanism 636, such as one or more LED outputs for visual indications of BLS device operation and for the control of visual effects. Visual indicators may include LED outputs controlling red, green, and blue hues of individual LEDs or a combined multi-hue LED device.

Controller 638 may additionally include wireless communications means, for example, using an onboard wireless transceiver 640 having an antenna, or alternatively, using an external wireless communications transceiver without loss of generality to the functionality of the BLS device 602. Wireless communications includes communications by Bluetooth Low Energy (BLE) to conserve battery power during operation, or alternatively near-field communications (NFC) protocol or other suitable protocol for the wireless communications between a pair of BLS devices or between a BLS device 602 and a portable control device.

The controller 638 may implement a Boolean Logic Controller (BLC) for operating a Boolean state machine, the BLS device operating autonomously in communication with a paired BLS device, a master BLS device, or a portable control device according to the modes of operation described herein.

The present disclosure provides, generally, computer and logic circuit-controlled devices configured to implement the methods and systems described above. Such devices may include central processing units (CPU) (e.g., processors) which may include or be in communication with memory (e.g., random-access memory, read-only memory, flash memory), electronic storage units (e.g., static RAM, memory stick, SDRAM modules), communication interfaces (e.g., network adapters, wireless adapters) for communicating with one or more other systems, and peripheral devices, such as cache, other memory, data storage and/or electronic display adapters, include touch-sensitive graphical display devices. Memory, storage units, interfaces, and peripheral devices are known to be communications with the CPU processors through communication buses, which may be a motherboard or a backplane of a computing or controller device. Computer or controller devices may be operatively coupled to a computer network (e.g. the internet) by one or more communications interfaces.

In one embodiment, the BLS device may house a printed circuit board (PCB). The PCB may include a processor and a memory for storing data and instructions for performing operations and functions of the BLS device. Instructions performed by the processor may be configured to implement a Boolean logic controller for initialization, charging, and to perform the EMDR functions as described in detail herein. The PCB may be configured to implement a Boolean logic state machine for control of BLS device functions. The PCB may be based, in some embodiments, on the ARDUINO™ hardware and software system, which may be integrated with, or integrate the functions of the BLS device. Pressure sensitive actuators may be configured to interact with the PCB to allow user control of settings of the BLS device and to control operations of the BLS device.

Computer processes implementing the control logic of the present disclosure may execute a sequence of machine-readable instructions, which can be embodied in a program or software. The instructions may be stored in a memory location. The instructions can be directed to the processor, which can subsequently program or otherwise configure the processor to implement methods of the present disclosure. Processors may be part of a circuit, such as an integrated circuit and one or more other components or modules of the computer systems may be included in a circuit, for example, in some cases, the circuits may be an application specific integrated circuit (ASIC).

Memory storage may store files, such as drivers, libraries and saved programs. Storage units may store user data, e.g., user preferences and user programs. Methods as described herein may be implemented by way of machine (e.g., CPU, processor) executable code stored on an electronic storage location of the computer or controller device. Machine executable or machine-readable code may be provided in the form of software. During use, the code may be executed by the processor, retrieved from the storage unit and stored on the memory for ready access by the processor. In some situations, machine-executable instructions may be stored directly to memory. Computer codes may be pre-compiled and configured for use with a machine have a processer adapted to execute the code or can be compiled during runtime. The code can be supplied in a programming language that can be selected to enable the code to execute in a pre-compiled or as compiled fashion.

As described herein, various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable updating of the software, for example, from a management server or host computer into the computer platform of an application server.

As used herein, unless restricted to non-transitory, tangible “storage” media, terms such as computer or machine “readable medium” refer to any medium that participates in providing instructions to a processor for execution. Machine readable medium may take many forms, including but not limited to, a tangible storage medium, a carrier wave medium or physical transmission medium. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) or a graphical user interface (GUI) for providing, for example, user interfaces associated with the system. Methods and systems of the present disclosure can be implemented by way of one or more algorithms. An algorithm can be implemented by way of software upon execution by a CPU/processor.

While certain embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions may occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure.

It is therefore contemplated that the disclosure shall also cover any such alternatives, modifications, variations or equivalents. It is intended that the following claims define the scope of the disclosure and that methods and structures within the scope of these claims and their equivalents be covered thereby.

A number of embodiments have been described. Nevertheless, it will be understood that various modifications may be made without departing from the spirit and scope of the disclosure. Accordingly, other embodiments are within the scope of the following claims.

What is claimed is:

1. A controller, comprising:
a memory; and
a processor configured to execute instructions stored in the memory to:
receive an input to initiate bilateral stimulation (BLS) stimuli;
generate parameters for the BLS stimuli based on the received input; and
cause BLS devices to output the BLS stimuli for a user of the BLS devices according to the generated parameters.

2. The controller of claim 1, wherein the processor is configured to execute the instructions to cause the BLS devices to output the BLS stimuli by causing the BLS devices to alternately output tactile stimulation via a vibration mechanism of the BLS devices according to the generated parameters.

3. The controller of claim 1, wherein the processor is configured to execute the instructions to cause the BLS devices to output the BLS stimuli by causing the BLS devices to alternately output visual stimulation via a light mechanism of the BLS devices according to the generated parameters.

4. The controller of claim 1, wherein the processor is configured to execute the instructions to cause the BLS devices to output the BLS stimuli by causing the BLS devices to alternately output audible stimulation via an audio output mechanism of the BLS devices according to the generated parameters.

5. The controller of claim 1, wherein generating the parameters includes generating at least one of a time synchronization of the BLS devices for the BLS stimuli, an alternation interval for the BLS stimuli, and an intensity level of the BLS stimuli.

6. The controller of claim 1, wherein the input includes session data for a real-time BLS session and the processor is configured to execute the instructions to:
generate the parameters utilizing the session data; and
cause the BLS devices to output the BLS stimuli for the real-time BLS session according to the generated parameters utilizing the session data.

7. The controller of claim 1, wherein the input includes predetermined session data for a pre-recorded BLS session and the processor is configured to execute the instructions to:
generate the parameters utilizing the predetermined session data; and
cause the BLS devices to output the BLS stimuli for the pre-recorded BLS session according to the generated parameters utilizing the predetermined session data.

8. The controller of claim 1, wherein the processor is configured to execute the instructions to:
receive biometric information about the user from a mobile device; and
generate the parameters for the BLS stimuli based on the received input and the biometric information.

9. The controller of claim 1, wherein the controller is included in a BLS device of the BLS devices.

10. A controller, comprising:
a memory; and
a processor configured to execute instructions stored in the memory to:
receive an input to initiate bilateral stimulation (BLS) stimuli, wherein the input includes predetermined session data for a pre-recorded BLS session;
generate parameters for the BLS stimuli utilizing the predetermined session data; and
cause BLS devices to output the BLS stimuli for a user of the BLS devices according to the generated parameters for the pre-recorded BLS session.

11. The controller of claim 10, wherein the processor is configured to execute the instructions to cause the BLS devices to output the BLS stimuli by causing the BLS devices to alternately output, for the pre-recorded BLS session, at least one of:
tactile stimulation via a vibration mechanism;
visual stimulation via a light mechanism; and
audible stimulation via an audio output mechanism.

12. The controller of claim 10, wherein the BLS devices include a primary BLS device and a secondary BLS device.

13. The controller of claim 12, wherein the processor is configured to execute the instructions to cause the BLS devices to output the BLS stimuli by transmitting an output command to the primary BLS device.

14. The controller of claim 13, wherein the primary BLS device is to transmit the output command to the secondary BLS device.

15. A method, comprising:
receiving, by a first controller associated with a first pair of bilateral stimulation (BLS) devices, an input to cause the first pair of BLS devices to initiate BLS stimuli, wherein the input includes session data for a BLS session;
generating, by the first controller, parameters for the BLS stimuli utilizing the session data; and
causing, by the first controller, the first pair of BLS devices to output BLS stimuli for a first user of the first pair of BLS devices according to the generated parameters for the BLS session.

16. The method of claim 15, wherein the method includes transmitting, by a portable control device, the input to the first controller.

17. The method of claim 16, wherein the method receiving, via a user interface of the portable control device, an input including the session data.

18. The method of claim 15, wherein the method includes transmitting, by a remote portable control device, the input to a second controller associated with a second pair of BLS devices to output BLS stimuli for a second user of the second pair of BLS devices, wherein the first pair of BLS devices and the second pair of BLS devices comprise a group of BLS devices.

19. The method of claim 18, wherein the session is a real-time group BLS session.

20. The method of claim 19, wherein the method includes causing, based on the input from the remote portable control device, the group of BLS devices to output BLS stimuli for the first user and the second user during the real-time group BLS session.
